(19) United States
(12) Patent Application Publication
(10) Pub. No.: US 2025/0090712 A1
(43) Pub. Date: Mar. 20, 2025

(54) DIFFUSER

(71) Applicant: MIRO CO., LTD., Incheon (KR)

(72) Inventors: Dong Jin Seo, Incheon (KR); Yong Ju Oh, Incheon (KR); Min Seok Kim, Incheon (KR)

(73) Assignee: MIRO CO., LTD., Incheon (KR)

(21) Appl. No.: 18/565,127

(22) PCT Filed: Jul. 27, 2023
(86) PCT No.: PCT/KR2023/010923

§ 371 Date: Nov. 29, 2023

(30) Foreign Application Priority Data
Aug. 23, 2022 (KR) 10-2022-0105693
Sep. 21, 2022 (KR) 10-2022-0119417

(51) Int. Cl. A61L 9/12

(52) U.S. Cl. CPC A61L 9/122; A61L 2209/134

(57) ABSTRACT

Provided is a diffuser equipped with an integrated cover that is opened and closed by an air current formed when a fan module is driven to control the spread of scent. The diffuser includes a body forming an exterior, a base inserted and fixed into the body and having a through-portion formed on one surface thereof, and an aroma container coupled to the base, wherein the base includes a cover rotatably provided on the through-portion, and an internal space of the body and an internal space of the base are selectively communicated with each other as the cover is rotated.

### CROSS-REFERENCE TO RELATED APPLICATION

This application is a national stage application of PCT/KR2023/010923 filed on Jul. 27, 2023, which claims the priority and benefit of Korean Patent Application No. 10-2022-0105693 filed on Aug. 23, 2022 and Korean Patent Application No. 10-2022-0119417 filed on Sep. 21, 2022, with the Korean Intellectual Property Office, the disclosure of which is incorporated herein by reference.

### BACKGROUND OF THE INVENTION

### 1. Field of the Invention

The present invention relates to a diffuser equipped with an integrated cover that is opened and closed by an air current formed when a fan is driven to control the spread of a scent.

### 2. Description of the Related Art

Diffusers are used in residential spaces, company offices, restrooms, restaurants, etc. to remove unpleasant odors, comfort users' minds and bodies, and change moods. Each user prefers different scents of diffusers, so many incense burners are manufactured and released, and users use different scents depending on time and place to change mood according to the situation.

Aroma diffusers are used to create a pleasant indoor environment and provide mental and physical comfort or for disease prevention and beauty purposes, and various aroma diffuser products, ranging from a product with an aromatic function of releasing essential oil components into the air through evaporation or vaporization due to wind or heat by diluting a certain amount of essential oil in water to a product with an aromatic function and humidifying function of changing water diluted with essential oil into water vapor and discharging the vapor into the air in order to maintain indoor humidity at a level suitable for the human body are available in the market.

In general, diffusers include a gas discharge type, a volatilization type, and a spray type depending on the method of providing a scent. The gas discharge type includes a component that sprays a scent using high pressure of gas, the volatilization type includes a component that spreads a scent by volatilizing perfume in a solid, liquid, or gel state contained in a container, and the spray type includes a component that physically presses and sprays liquid perfume.

The gas discharge and spray type diffusers require a separate component for spraying a scent, and noise occurs from the component for spraying a scent when the diffuser operates, which may interfere with the user's mental and physical stability and mood change.

In addition, in the case of the volatilization type, the aforementioned problem still exists because a separate driving unit should be provided to control spraying of a scent by adjusting volatilization of perfume in a solid, liquid, or gel state or the degree of volatilization depending on whether the diffuser operates.

### SUMMARY

Therefore, the present invention aims to solve the aforementioned problems.

An aspect of the present invention may provide a diffuser having a structure that may emit a scent only through an air current formed by a fan when the diffuser operates.

Another aspect of the present invention may provide a diffuser having a structure that may reduce noise occurring when the diffuser operates.

Another aspect of the present invention may provide a diffuser having a structure that may block a flow path based on structural characteristics without separate electronic control after a fan stops operating.

An aspect of the present invention may also provide a diffuser including: a body forming an exterior; a base inserted and fixed into the body and having a through-portion formed on one surface thereof; and an aroma container coupled to the base, wherein the base includes a cover rotatably provided on the through-portion, and an internal space of the body and an internal space of the base are selectively communicated with each other as the cover is rotated.

The base further may include a coupling portion, the coupling portion may be formed at a position facing the through-portion, and the aroma container may be coupled to the base through the coupling portion.

The diffuser may further include: a fan coupled to cover a first portion of the through-portion to form flow of air.

The diffuser may further include: a flow path member provided inside the body to guide the flow of air formed by the fan, wherein the flow path member is provided at a position in communication with the second portion of the through-portion on one surface of the base.

The cover may include a first cover, a second cover, and a rotating shaft, the first cover may be provided at a position corresponding to at least a portion of the first portion, the second cover may be provided at a position corresponding to the second portion or a remaining portion of the second portion and the first portion, and the rotating shaft may be formed between the first cover and the second cover.

An area formed by the first cover may be greater than an area formed by the second cover.

A weight formed by the first cover may be less than a weight formed by the second cover.

The second cover may have a step formed at an end, and the through-portion may include a rotation preventing portion contacting the step of the second cover to limit rotation of the second cover.

The second cover may include a protrusion preventing one surface of the second cover from colliding or being in surface-contact with an inner surface of the flow path member when the second cover rotates.

The diffuser may further include a circuit board coupled to the base, and the circuit board may be located between the fan and an inner surface of the body.

The body may further include: a hole connecting the outside of the body and the flow path member through one surface of the body; and an opening and closing member selectively opening and closing the hole.

An aspect of the present invention may also provide a diffuser including: a body forming an exterior of the diffuser and allowing fluid to be discharged to one side thereof and an aroma container accommodating fluid therein and supplying the fluid to the inside of the body; and a base inserted and fixed into the body, allowing the aroma container to be coupled to one side thereof, and including the through-portion formed on the other side thereof, wherein the base includes a cover rotatably provided in the through-portion, and the inside of the body and the inside of the base selectively communicate with each other as the cover is rotated.

Each feature of the embodiments described above may be implemented in combination with other embodiments as long as it is not contradictory or exclusive to other embodiments.

According to various embodiments of the present invention, emitting of a scent by the diffuser may be controlled solely by an air current generated from the fan without a separate drive motor.

In addition, noise occurring when the diffuser is driving may be reduced, thereby creating a pleasant indoor environment for users and providing mental and physical stability.

In addition, users may easily remove the diffuser to clean internal components, thereby facilitating maintenance and repair the diffuser.

The effects of the present invention are not limited to those described above, and other effects not mentioned will be clearly recognized by those skilled in the art from the description below.

### BRIEF DESCRIPTION OF DRAWINGS

FIG. 1 is a diagram illustrating a diffuser according to an embodiment of the present invention.

FIG. 2 is a view illustrating one side of FIG. 1.

FIG. 3 is a diagram illustrating part of an internal configuration of a diffuser according to an embodiment of the present invention.

FIG. 4 is a diagram illustrating a cross-section of a diffuser according to an embodiment of the present invention.

FIG. 5 is a diagram illustrating a configuration of a diffuser according to an embodiment of the present invention.

FIG. 6 is a diagram illustrating a coupling relationship between a diffuser and an aroma container according to an embodiment of the present invention.

FIG. 7 is a diagram illustrating a base and a fan module according to an embodiment of the present invention.

FIG. 8 is a diagram illustrating a base and a cover according to an embodiment of the present invention.

FIG. 9 is a diagram illustrating a cover according to an embodiment of the present invention.

FIG. 10 is a diagram illustrating rotation of a cover when a diffuser operates according to an embodiment of the present invention.

### DETAILED DESCRIPTION

Hereinafter, embodiments of the present invention are described with reference to the accompanying drawings. The following description is provided to aid in the comprehensive understanding of methods, devices, and/or systems disclosed in the particularities. However, the following description is merely exemplary and not provided to limit the present invention.

In the following description of the present invention, a detailed description of known functions and configurations incorporated herein will be omitted when it would make the subject matter of the present invention unclear. The terms used in the present specification are defined in consideration of functions used in the present invention, and may be changed according to the intent or conventionally used methods of operators, and users. Accordingly, definitions of the terms should be understood on the basis of the entire description of the present specification. Terms used in the following description are merely provided to describe embodiments of the present invention and are not intended to be limiting of the inventive concept. As used herein, the singular forms “a,” “an” and “the” are intended to include the plural forms as well, unless the context clearly indicates otherwise. It will be further understood that the terms “comprises” or “has” when used in this specification, specify the presence of stated features, integers, steps, operations, elements, or a portion or combination thereof, but do not preclude the presence of addition of one or more other features, integers, steps, operations, elements, or a portion or combination thereof.

In describing the elements of the present invention, terms, such as first, second, A, B, (a), (b), etc., may be used. Such terms are used for merely discriminating the corresponding elements from other elements and the corresponding elements are not limited in their essence, sequence, or precedence by the terms.

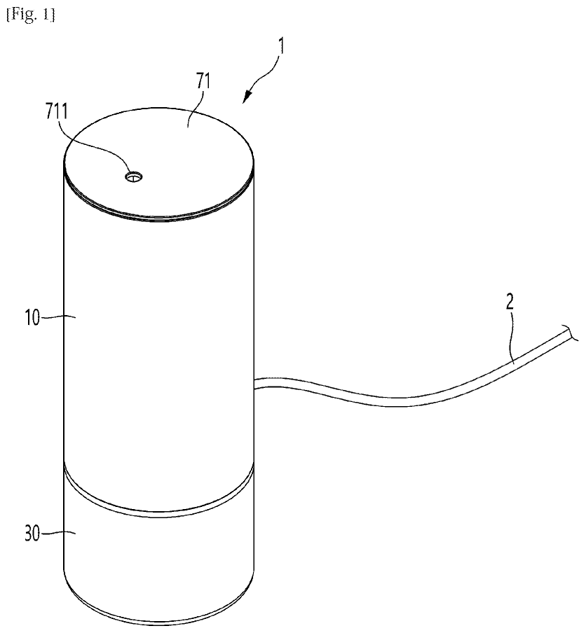

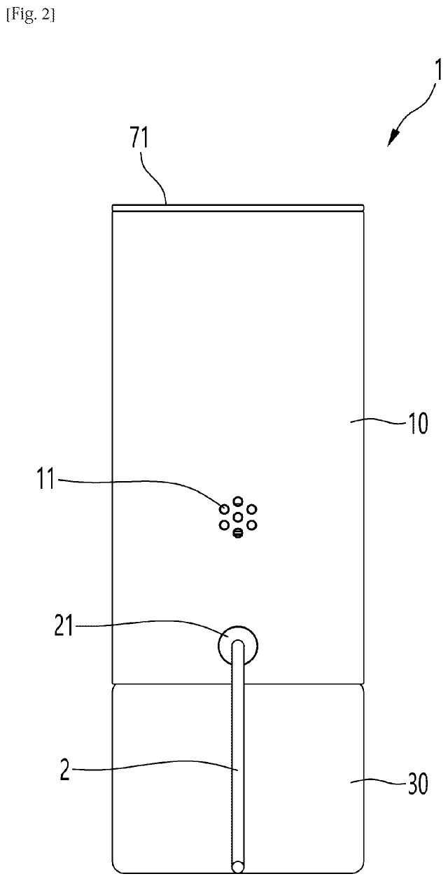

FIG. 1 is a diagram illustrating a diffuser according to an embodiment of the present invention, and FIG. 2 is a diagram illustrating one side of FIG. 1.

Hereinafter, descriptions are given with reference to FIGS. 1 and 2.

A diffuser 1 according to an embodiment of the present invention may include a body 10 and an aroma container 30. The body 10 may form the exterior of the diffuser 1, may be provided in a cylindrical shape, and may form an accommodating space 10s therein. The body 10 may have various shapes forming the internal accommodating space 10s.

A power supply line 2 may be connected to one side of the body 10 through a connecting member 21, so that power for operating the diffuser 1 may be supplied from the outside. The connecting member 21 may be formed of a rubber material to prevent coating from peeling off or breaking due to bending of the power supply line 2. In addition, the connecting member 21 may assist in a rotation radius of the power supply line 2 in order to minimize restrictions on an installation location and installation direction by the power supply line 2 when selecting the installation location of the diffuser 1.

An intake port 11 may be formed on one side surface of the body 10. Referring to FIG. 2, the connecting member 21 and the intake port 11 may be formed on the same side surface. The intake port 11 or the connecting member 21 may be formed on different side surfaces of the body 10.

A discharge port 711 may be formed on an upper surface 71 of the body 10. The upper surface 71 may be formed integrally with the body 10 to form an upper surface of the body 10, or the upper surface 71 may be provided independently from the body 10 and be detachable from the body 10.

The discharge port 711 may communicate with a flow path member 70, which will be described below, so that perfume contained in the aroma container 30 may be discharged through the discharge port 71 when the diffuser 1 operates. Perfume may refer to at least one of various materials for emitting a scent, such as liquid perfume and solid perfume.

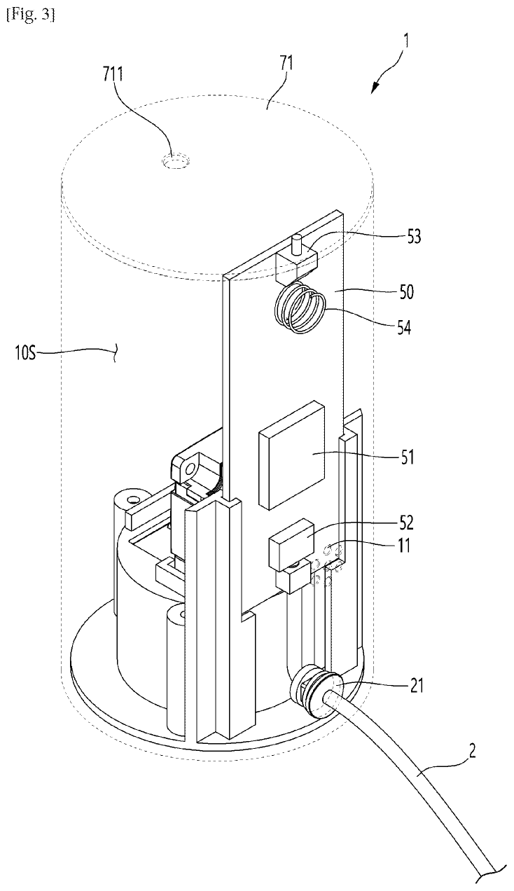

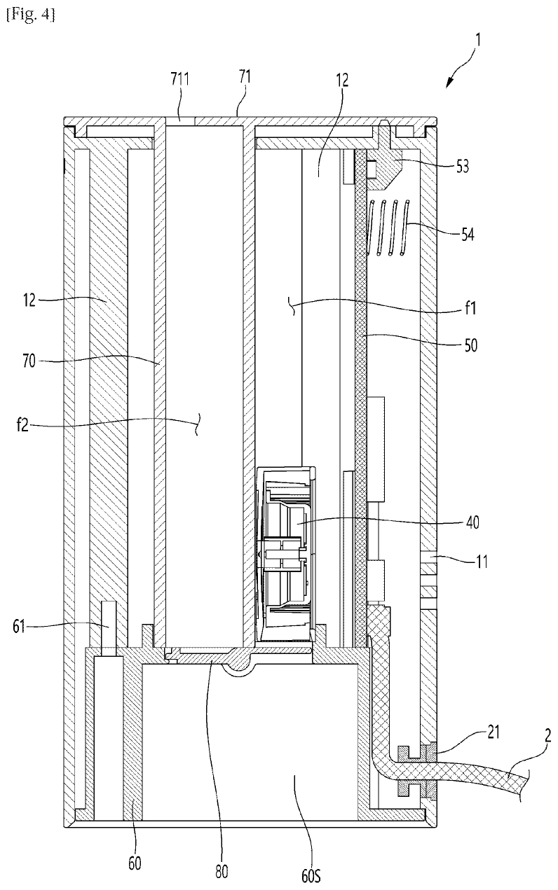

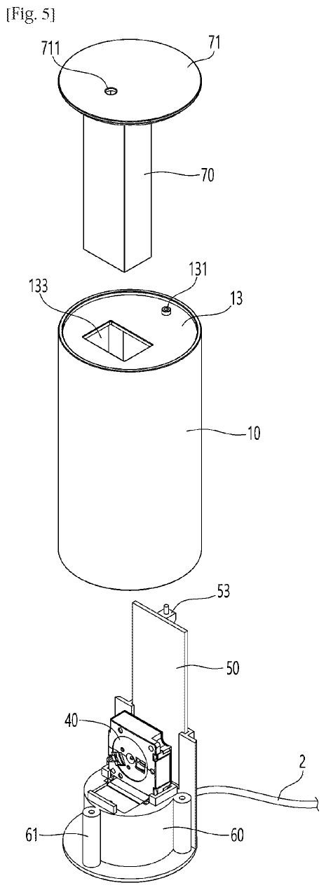

FIG. 3 is a diagram illustrating part of an internal configuration of a diffuser according to an embodiment of the present invention, FIG. 4 is a diagram illustrating a cross-section of a diffuser according to an embodiment of the present invention, and FIG. 5 is a diagram a configuration of a diffuser according to an embodiment of the present invention.

Hereinafter, descriptions will be given with reference to FIGS. 3 to 5.

The internal space 10s of the body 10 may be provided with components for emitting a scent of the diffuser 1 and components for operation control of the diffuser 1. More specifically, the internal space 10s of the body may be provided with various components for a configuration and operation control of the diffuser 1 including a fan module 40, a circuit board 50, a base 60, a flow path member 70, and a cover 80.

The fan module 40 may include a blade that rotates about one axis, a rotating shaft of the blade, and a motor for providing rotational power to the blade. The fan module 40 may operate upon receiving power through the power supply line 2 and may be controlled through a control module 51 provided in the circuit board 50.

The base 60 may be inserted into and fixed to the body 10. More specifically, the base 60 may be fitted through a coupling member 61 of the base 60 and a coupling member 12 of the body 10. In order to firmly couple the base 60 and the body 10, a plurality of coupling members 61 of the base and a plurality of coupling members 12 of the body may be provided at corresponding positions. The base 60 may form an internal space 60s therein in a longitudinal direction. The internal space 60s may selectively communicate with a flow path f2 through rotation of the cover 80, and details thereof will be described below.

The circuit board 50 may be coupled to one surface of the base 60. When the circuit board 50 is coupled to one surface of the base 60, it may be located between the inner surface of the body 10 and the fan module 40. More specifically, the fan module 40 may be provided at a position corresponding to the intake port 11 formed on the side surface of the body 10, and the circuit board 50 may be located between the fan module 40 and the intake port 11, thereby preventing noise occurring from the fan module 40 from being exposed to the outside when the diffuser 1 operates.

The fan module 40 and the circuit board 50 may be coupled to the base 60. More specifically, the fan module 40 and the circuit board 40 may be coupled to the upper surface of the base 60 and provided in the internal space of the body 10, a fan module coupling portion 62 to which the fan module 40 is coupled may be formed on the upper surface of the base 60, and a guide member 63 to which the circuit board 50 is coupled may be formed.

A groove 631 may be formed in the guide member 63, and both side surfaces of the circuit board 50 may be inserted and fixed along the groove 631.

In this structure, after external air is introduced through the intake port 11, an air current formed by the fan module 40 may move along a flow path f1 provided with the fan module 40 through an end of the guide member 63, that is, both ends of the circuit board 50, pass through an internal space 60s of the base 60 according to rotation of the cover 80, pass through the flow path f2 formed by the flow path member 70, and then be discharged to the outside of the diffuser 1 through the discharge port 711.

As described above, the upper surface 71 may be formed integrally with the body 10 or may be formed independently. The upper surface 71 of the present embodiment may be an independent component of the body 10, and the flow path member 70 forming the flow path f2 in the longitudinal direction of the body 10 may be connected to the upper surface 71, and the discharge port 711 penetrating through the flow path f2 may be formed.

The flow path member 70 may be provided in the internal space 10s of the body 10 and may guide flow of air formed by the fan module 40. The flow path member 70 may be formed integrally with the upper surface 711. The upper surface 711 may be in contact with a contact surface 13 when coupled to the body 10 to form the upper surface of the exterior of the diffuser 1.

A through-portion 133 and a hole 131 may be formed on an upper contact surface 13 of the body 10. When the upper surface 71 is coupled to the body 10 through the through-portion 133, the flow path member 70 may be introduced into the internal space 10s of the body 10.

The hole 131 may be formed at a position corresponding to a light emitting member 53 connected to the circuit board 50, and the light emitting member 53 may serve as an indicator that may operate depending on an operating state of the diffuser 1.

The upper surface 71 may include an opening and closing member (not shown) that selectively opens and closes the discharge port 711. The opening and closing member may be provided in various forms and, for example, may open and close the discharge port 711 by sliding. The amount of discharged fluid may be controlled depending on the degree to which the discharge port 711 is opened and closed. In addition, the opening and closing member may open and close the discharge port 711 in conjunction with the driving of the fan module 40. In this case, the aforementioned control may be performed by the control module 51.

In the structure of the flow path member 70 described above, when a user separates the diffuser 1 for maintenance of the diffuser 1, the upper surface 71 may be separated from the body 10, and the flow path member 70 connected to the upper surface 71 may be easily separated from the body 10 so that contaminants, oil, etc. accumulated on the inner surface of the flow path member 70 may be easily removed.

The circuit board 50 may include various components for controlling the operation of the diffuser 1. As an example, the circuit board 50 may include the control module 51, a sensor module 52, the light emitting member 53, and a connecting member 54.

The sensor module 52 may measure an external temperature, humidity, air quality, etc. The connecting member 54 may electrically connect the side surface of the body 10 to the circuit board 50 so that the user may control the operation of the diffuser 1 through a portion of the side surface of the body 10 connected to the connecting member 54. An operation control status of the diffuser 1 may be displayed to the user through the light emitting member 53. In this case, the operation control status of the diffuser 1 may be comprehensively displayed in consideration of external air conditions measured by the sensor module 52.

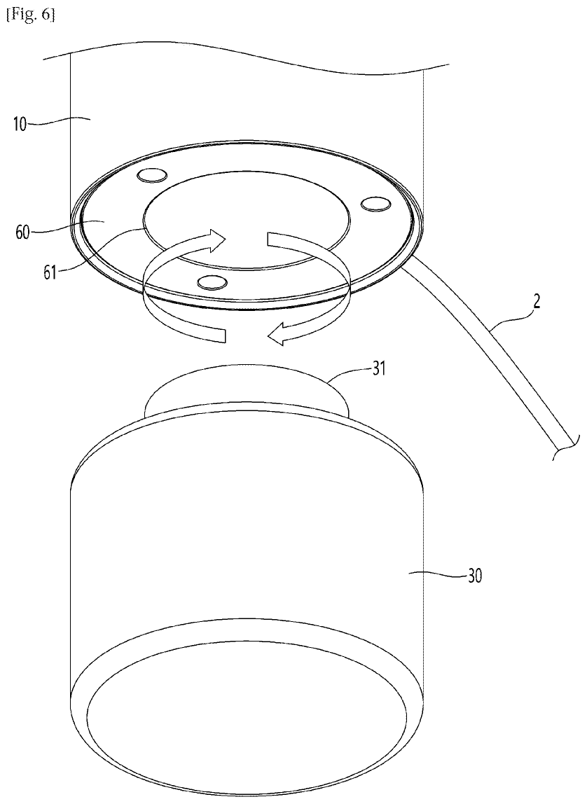

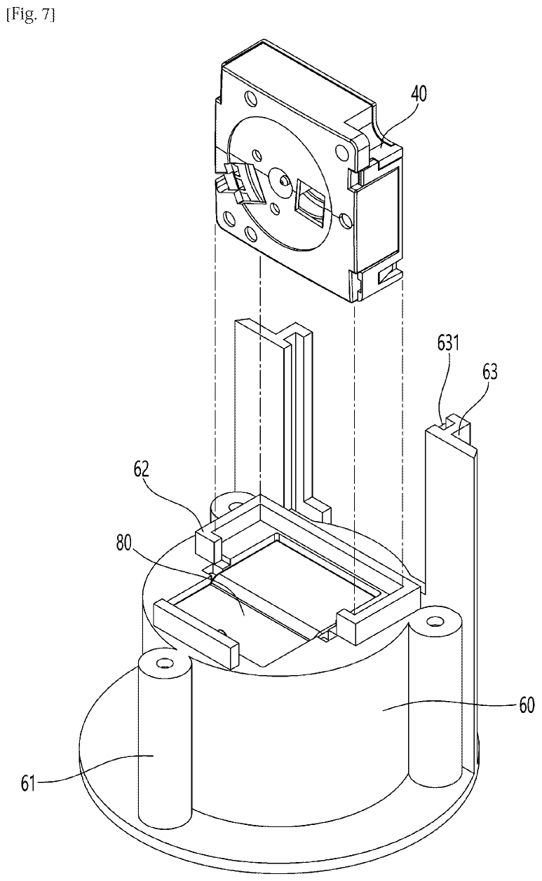

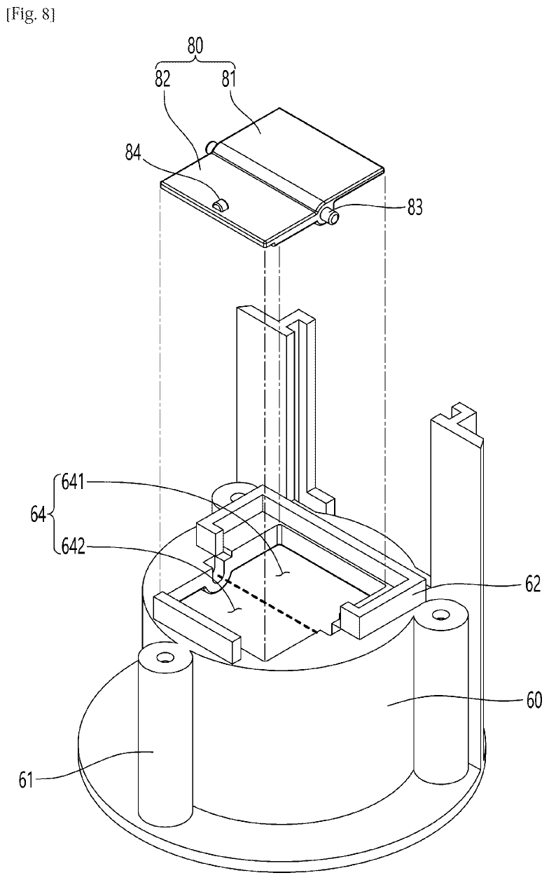

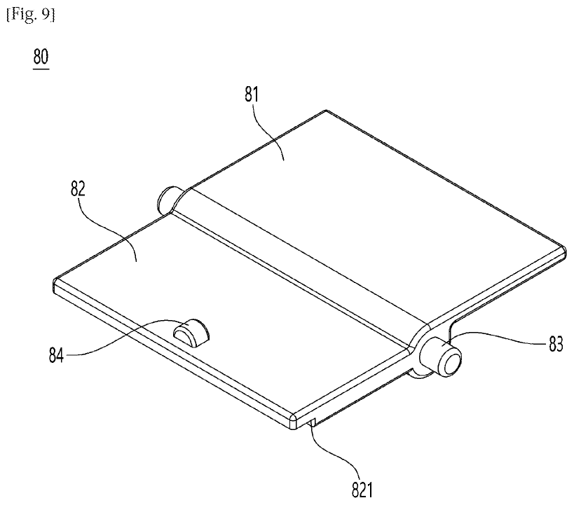

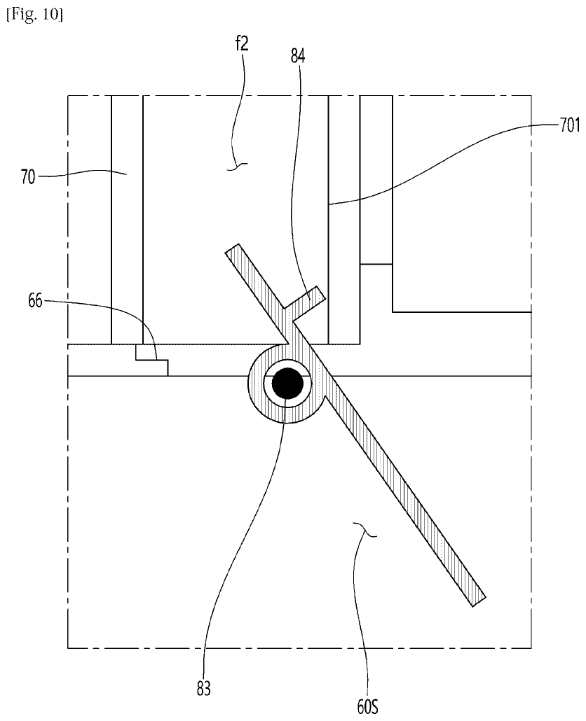

FIG. 6 is a diagram illustrating a coupling relationship between a diffuser and an aroma container according to an embodiment of the present invention, FIG. 7 is a diagram illustrating a base and a fan module according to an embodiment of the present invention, FIG. 8 is a diagram illustrating a base and a cover according to an embodiment of the present invention, FIG. 9 is a diagram illustrating a cover according to an embodiment of the present invention, and FIG. 10 is a diagram illustrating rotation of a cover when operating a diffuser according to an embodiment of the present invention.

Hereinafter, descriptions will be given with reference to FIGS. 6 to 10.

The through-portion 64 may be formed on one surface of the base 60. The fan module coupling portion 62 and the guide member 63 may be formed to protrude from one surface of the base 60 in which the through-portion 64 is formed. The coupling portion 61 may be formed on the other side of the base 60. The base has a hollow shape extending by a predetermined length from one surface to the other side, thereby forming the space 60s therein.

The space 60s may selectively communicate by the cover 80 with the flow path f2 formed by the flow path member 70 and the flow path f1 provided with the fan module 40 and may communicate with the aroma container 30 as the aroma container 30 is coupled to the coupling portion 61.

A coupling portion 31 is formed in the aroma container 30 and may be rotatably coupled to the coupling portion 61 of the base 60. In order for the aroma container 30 to be rotatably coupled to the other side of the base 60, threads engaging with each other may be formed in the coupling portion 31 of the aroma container 30 and the coupling portion 61 of the base 60, respectively.

The aroma container 30 may be coupled to a lower portion of the base 60 so that scent may spread into the internal space 60s of the base 60. As a rotational coupling structure of the aroma container 30 and the base 60, various coupling structures for spreading the scent contained in the aroma container 30 into the internal space 60s of the base 60 may be adopted, for example.

The cover 80 may be rotatably coupled to the through-portion 64. By rotating the cover 80, the internal space 10s of the body and the internal space 60s of the base may selectively communicate with each other. When the aroma container 30 is coupled to the base 60, the internal space 10s of the body 10 and the internal space 60s of the base 60 may communicate with each other as the through-portion 64 is selectively opened and closed according to rotation of the cover 80. That is, the through-portion 64 may be selectively opened and closed according to the rotation of the cover 80, and accordingly, the aroma container 30 connected to the other side of the base 60 may selectively communicate with the internal space 10s of the body through the through-portion 64.

The cover 80 may be rotated by the air current formed by the fan module 40. More specifically, the fan module 40 may be coupled to a position corresponding to at least a portion of a first portion 641 of the through-portion 64. More specifically, the fan module 40 may be coupled to the fan module coupling portion 62 formed on one surface of the base 60, while covering the first portion 641 of the through-portion 64, thereby forming the air current. That is, the fan module coupling portion 62 may be formed to protrude along at least a portion of the circumference of the first portion 641 of one surface of the base 60.

The first portion 641 may refer to a portion of the through-portion 64 formed on one surface of the base 60. The air current formed by the fan module 40 may rotate the cover 80 rotatably coupled to the through-portion 64, while moving toward the first portion 641.

The flow path member 70 may be provided at a position in communication with the second portion 642 of the through-portion 64 on one surface of the base 60. The second portion 642 may refer to at least a portion of the through-portions 64 formed on one surface of the base 60, excluding the first portion 641.

The cover 80 may include a first cover 81, a second cover 82, and a rotating shaft 83. The cover 80 may be coupled to a position corresponding to the through-portion 64 on the base 60 as the rotating shaft 83 of the cover is coupled to the grooves 65 formed on both sides of the through-portion 64.

The first cover 81 may be provided at a position corresponding to at least a portion of the first portion 641, and the second cover 82 may be provided at a second portion 642 or at a position corresponding to a remaining portion of the second portion 642 and the first portion 641.

That is, when the cover 80 is coupled to the base 60, the area formed by the first cover 81 may be provided at a position covering a portion corresponding to at least a portion of the first portion 641 of the through-portion, and the area formed by the second cover 82 may be provided at a position covering the other remaining portion excluding the portion in which the through-portion 64 is covered by the firs cover 81. Also, the rotating shaft 83 may be formed between the first cover 81 and the second cover 82.

According to the structure described above, since at least a portion of the first cover 81 is provided at a position facing the fan module 40 coupled to the fan module coupling portion 62, the air current formed by driving the fan module 40 may push the first cover 81 so that the first cover 81 may be rotated based on the rotating shaft 83.

That is, the first cover 81 may be rotated toward the internal space 60s of the base by the air current formed by the fan module 40. When the aroma container 30 is coupled to the base 60, based on the aroma container 30, the first cover 81 may be rotated in a direction toward the aroma container 30 by the air current formed by the fan module 40, so that the air current formed by the fan module 40 may be guided toward the aroma container 30.

Also, the second cover 82 may be rotated toward the flow path f2 formed by the flow path member 70 in conjunction with the first cover 81. As described above, the flow path member 70 is provided at a position facing the second portion 642 of the through-portion, so that the second cover 82 may rotate in a direction toward an inner surface 701 of the flow path member, so that the flow path f2 and the internal space 60s of the base may communicate with each other.

When the aroma container 30 is coupled to the base 60, based on the aroma container 60, the second cover 82 may be rotated toward the inner surface 701 of the flow path member in conjunction with the first cover 81, so that the air current formed by the fan module 40 may be guided toward the flow path f2 through the aroma container 30.

It is preferable that the area formed by the first cover 81 is formed to be larger than the area formed by the second cover 82. The reason is because, since the first cover 81 is pushed and rotated by the air current formed by the fan module 40, in order for the first cover 81 to be pushed more easily, it is advantageous to expand the contact area with the air current formed by the fan module 40.

In addition, it is preferable that the weight of the first cover 81 is lighter than that of the second cover 82. This is because, in order for the second cover 82 to rotate more easily in the direction toward the internal space 60s of the base or the aroma container 30, it is advantageous for the weight of the second cover 82 to be greater than the weight of the first cover 81.

In addition, as an example to realize a weight difference between the first cover 81 and the second cover 82, the thickness formed by the first cover 81 may be less than the thickness formed by the second cover 82.

That is, after the driving of the fan module 40 is stopped, in order for the cover 80 to rotate to easily block the internal space 10s of the body and the internal space 60s of the base, it is advantageous for the weight of the second cover 82 is greater than the weight of the first cover 81. Based on the aroma container 30, in order for the cover 80 to rotate to block communication between the flow paths f1 and f2 and the aroma container 30 after the fan module 40 stops driving, it is advantageous for the weight of the second cover 82 to be greater than the weight of the first cover 81.

Meanwhile, the second cover 82 has a step 821 formed at an end portion thereof, and a rotation preventing portion 66 contacting the step 821 of the second cover 82 to limit rotation of the second cover 82 may be formed at an end portion of the through-portion 64.

That is, the step 821 formed on the second cover 82 contacts the rotation preventing portion 66, so that, after the fan module 40 stops driving, the degree to which the cover 80 is rotated from the inner side of the flow path member toward the internal space 60s of the base or the aroma container 30 may be limited.

Also, the second cover 82 may further include a protrusion 84 that prevents one surface of the second cover 82 from colliding with the inner surface 701 of the flow path member. The protrusion 84 may prevent noise from occurring when the second cover 82 collides with the inner surface 701 of the flow path member when rotating toward the inner side of the flow path member in conjunction with the rotation of the first cover 81 during driving of the fan module 40. In addition, the protrusion 84 may prevent damage to the second cover 82 by preventing the second cover 82 from colliding with the inner surface 701 of the flow path member.

In addition, the protrusion 84 may prevent one surface of the second cover 82 from being in surface-contact with the inner surface 701 of the flow path member. Since the flow path member 70 forms the flow path f2 through which fluid moves, fluid having a predetermined viscosity may accumulate on the inner surface 701 of the flow path member.

If the second cover 82 rotates to be in surface-contact with the inner surface 701 of the flow path member according to the operation of the diffuser 1, the operation of the diffuser 1 may be stopped due to contaminants having a predetermined viscosity accumulated on the inner surface 701 of the flow path member, and then, a state in which one surface of the second cover 82 is in contact with the inner surface 701 of the flow path member may be maintained. Therefore, it is desirable to prevent one surface of the second cover 82 from being in surface-contact with the inner surface 701 of the flow path member through the protrusion 84.

Although exemplary embodiments of the present invention have been illustrated and described hereinabove, the present invention is not limited to the aforementioned specific exemplary embodiments, but may be variously modified by those skilled in the art to which the present invention pertains without departing from the scope and spirit of the present invention as disclosed in the accompanying claims. These modifications should also be understood to fall within the scope of the present invention.

What is claimed is:

1. A diffuser comprising:
a body forming an exterior;
a base inserted and fixed into the body and having a through-portion formed on one surface thereof; and
an aroma container coupled to the base,
wherein the base includes a cover rotatably provided on the through-portion, and
an internal space of the body and an internal space of the base are selectively communicated with each other as the cover is rotated.

2. The diffuser of claim 1, wherein
the base further includes a coupling portion,
the coupling portion is formed at a position facing the through-portion, and the aroma container is coupled to the base through the coupling portion.

3. The diffuser of claim 1, further comprising:
a fan module coupled to a position corresponding to at least a portion of a first portion of the through-portion on one surface of the base to form flow of air.

4. The diffuser of claim 3, further comprising:
a flow path member provided inside the body to guide the flow of air formed by the fan module,
wherein the flow path member is provided at a position in communication with the second portion of the through-portion on one surface of the base.

5. The diffuser of claim 4, wherein
the cover includes a first cover, a second cover, and a rotating shaft,
the first cover is provided at a position corresponding to at least a portion of the first portion,
the second cover is provided at a position corresponding to the second portion or a remaining portion of the second portion and the first portion, and
the rotating shaft is formed between the first cover and the second cover.

6. The diffuser of claim 5, wherein an area formed by the first cover is greater than an area formed by the second cover.

7. The diffuser of claim 5, wherein a weight formed by the first cover is less than a weight formed by the second cover.

8. The diffuser of claim 5, wherein
the second cover has a step formed at an end, and
the through-portion includes a rotation preventing portion contacting the step of the second cover to limit rotation of the second cover.

9. The diffuser of claim 5, wherein the second cover includes a protrusion preventing one surface of the second cover from being in surface-contact with an inner surface of the flow path member when the second cover rotates.

10. The diffuser of claim 4, wherein the body further includes:
a discharge port connecting the outside of the body and the flow path member through one surface of the body; and
an opening and closing member selectively opening and closing the discharge port.
